(19) United States
(12) Patent Application Publication
(10) Pub. No.: US 2024/0374530 A1
(43) Pub. Date: Nov. 14, 2024

(54) EXOSOMES FROM COFFEE, GINKGO-BILOBA AND GINSENG

(71) Applicant: AYE EXOCURE TIBBI ÜRÜNLER SANAYI VE TICARET ANONIM SIRKETI, Çankaya/Ankara (TR)

(72) Inventors: Ahmet Yasar ESMEKAYA, Çankaya/Ankara (TR); Meriç Arda ESMEKAYA, Çankaya/Ankara (TR)

(21) Appl. No.: 18/690,610

(22) PCT Filed: May 24, 2023
(86) PCT No.: PCT/TR2023/050468

§ 371 Date: Mar. 8, 2024

(30) Foreign Application Priority Data
May 25, 2022 (TR) 2022/008499

(51) Int. Cl. A61K 9/51; A61K 36/16; A61K 36/258

(52) U.S. Cl. CPC A61K 9/5176; A61K 36/16; A61K 36/258; A61K 36/742

(57) ABSTRACT

Disclosed is the obtaining of exosomes from coffee, ginkgo-biloba, and ginseng plants and making tablets from the exosomes. The use of plant-derived exosomes obtained from coffee, ginkgo-biloba, and ginseng, with high bio-compatibility, cellular uptake, stability, and much more ability to penetrate the blood-brain barrier is also disclosed in the treatment of Alzheimer's disease, cognitive dysfunction, forgetfulness, and poor concentration disorders.

### TECHNICAL FIELD

The invention relates to obtaining exosomes from coffee, ginkgo-biloba and ginseng plants and making tablets from them.

The invention relates to the use of plant-derived exosomes obtained from coffee, ginkgo-biloba and ginseng which have high bio-compatibility, cellular uptake, stability and much more ability to penetrate through the blood-brain barrier in the treatment of Alzheimer's disease and the reduction of symptoms; in the treatment of cognitive dysfunction, forgetfulness and poor concentration disorders.

### Known State of the Art

Exosomes are natural nanoparticles of 30-150 nm size that are secreted by almost all cells. These nanoparticles have a membrane with double lipid layers and proteins bound to the membrane. In addition, they contain materials such as miRNA, DNA, proteins that carry the properties of the cell from which they originate. Exosomes originating from a particular cell have natural targeting potential due to the proteins contained in their membranes. Since genetic materials can change the phenotypic and genotypic characteristics of the targeted cell, there are limitations and disadvantages in the use of exosomes obtained from human cells. For this reason, research on plant-derived exosomes has recently intensified.

Plant-derived exosomes are small vesicles released by multi-vascular bodies mainly for communication between cells and regulating immunity against pathogen attack. It has been shown in studies conducted in literature that these nanoparticles are absorbed by intestinal macrophages and can exhibit interspecies communication by inducing multiple cytokines. The importance of plant-derived exosomes stems from their biomolecule content (lipids, proteins and miRNAs), their easy internalization by mammalian cells, their anti-inflammatory, immunomodulatory and regenerative properties. Plant-derived exosomes have the advantages of large-scale production, low toxicity, reduced immunogenicity, efficient cellular uptake, high biocompatibility and stability. Due to these characteristics, potential applications in medical treatments arouse curiosity, and in-vitro and in-vivo studies are being conducted in this field.

Alzheimer's Disease, the most common type of dementia, which is characterized by neuritic plaques and neurofibrillary tangles as a result of the accumulation of Aβ peptides in the medial temporal lobe and neocortical structures and is defined as a slow-progressing neurodegenerative disease. Although research is continuing on whether Aβ plaques are the cause or result of Alzheimer's disease, it is thought that the increased toxic effect of overproduction and accumulation of Aβ peptides is the primary cause of neuro-degeneration and cognitive impairment.

Coffee is one of the most widely consumed beverages worldwide. Coffee contains many bioactive compounds such as polyphenols, especially chlorogenic acid (CGA), caffeic acid, alkaloids (caffeine and trigonelline) and diterpenes (cafestol and kahweol). It has been shown in studies conducted in the literature that coffee consumption reduces the risk of developing neurodegenerative diseases such as Alzheimer's, Parkinson's and dementia. In experimental animal and human studies, it has been shown that various coffee extracts can alleviate the symptoms of Alzheimer's disease.

Clinical studies support the use of coffee to stimulate the production of brain-derived neurotrophic factor (BDNF). BDNF has powerful effects on the brain and central nervous system. By regulating the development and differentiation of new neurons, BDNF ensures the survival of neurons and supports balanced mood. BDNF production decreases with age and is observed at low levels in patients diagnosed with Alzheimer's disease. In a study, it was shown that a single dose of encapsulated coffee concentrate increased peripheral serum BDNF by 91% in the 60th minute and by 66% in the 120th minute compared to initial levels.

Ginseng is traditionally used in the treatment of many diseases in oriental medicine (oriental traditional medicine). In studies conducted in the literature, it has been shown that the symptoms of Alzheimer's disease may be alleviated with the intake of ginseng extract and may cause an increase in cognitive functions. Ginseng extracts are known to contain biologically active ingredients that can prevent cognitive disorders. It is stated that the biologically active chemicals in ginseng extracts can prevent cognitive disorders by reducing the formation and aggregation of AB. There are also studies in the literature showing that these extracts improve AB-induced mitochondrial damage and contribute to the reduction of anti-apoptotic protein levels.

Park et al. reported in their study an improvement in cognitive function due to Ginseng intake in volunteers with cognitive disorders.

Ginkgo-biloba is also used as a standardized extract for the treatment of various problems such as concentration problems, memory, confusion, depression, anxiety, dizziness, tinnitus and headaches. The main use of ginkgo-biloba is in the treatment of cerebral dysfunction. Ginkgo-biloba is recommended to slow the progression of neurodegenerative disorders such as age-related cognitive decline, Alzheimer's disease and other types of dementia.

Due to the large size of the extracts obtained from coffee, ginkgo-biloba and ginseng, their potential to penetrate through the blood-brain barrier is low. These properties of extracts obtained from coffee, ginkgo-biloba and ginseng limit the effects of these extracts in the treatment of Alzheimer's disease.

The effects of coffee, ginkgo-biloba and ginseng on treatment of Alzheimer's disease and the activities of the cognitive functions and concentration have been investigated using essences and extracts. However, a study on their use at the exosome level is not included in the literature and there are no patents available in this field. It is obvious that coffee, ginkgo-biloba and ginseng-derived exosomes, which provide an innovative perspective on the solution of Alzheimer's disease other than conventional methods, will be a new source of hope for people struggling with the disease in terms of preventing the formation of Aβ plaques, which are a pathological symptom of this disease. In this context, the use of exosomes obtained from coffee, ginkgo-biloba and ginseng for the first time and the fact that it considers the possible treatment of Alzheimer's disease in this context distinguishes it from other patents derived from coffee, ginkgo-biloba and ginseng for the treatment of this disease.

As a result, due to the above-mentioned negativities and due to the inadequacy of existing solutions on the subject, it has become necessary to make an improvement in the relevant technical field.

### BRIEF DESCRIPTION OF THE INVENTION

The present invention relates to exosomes obtained from coffee, gingko-biloba and ginseng, which meet the requirements mentioned above, eliminate all the disadvantages and provide some additional advantages.

The primary object of the invention is to provide the reduction of the symptoms of the disease in Alzheimer's patients and its treatment by using exosomes obtained from coffee, gingko-biloba and ginseng.

The primary object of the invention is to provide the treatment of cognitive dysfunction, dysmnesia and concentration difficulties by using exosomes obtained from coffee, gingko-biloba and ginseng.

Another object of the invention is to provide for the use of natural products with very low toxicity and side effects instead of synthetic molecules in the treatment of Alzheimer's disease and to increase cognitive functions and concentration.

Another object of the invention is to enable exosomes obtained from coffee, gingko-biloba and ginseng to penetrate much more through the blood-brain barrier due to their small size.

In order to fulfil the objectives described above, the invention relates to the use of coffee, ginkgo-biloba and ginseng plants in obtaining of exosomes.

In order to fulfil the objectives described above, the invention comprises the use of exosomes obtained from coffee, ginkgo-biloba and ginseng plants in the treatment of Alzheimer's disease, cognitive dysfunction, dysmnesia (forgetfulness) and concentration difficulty disorders.

The invention is plant-derived exosomes which is comprise their use in the treatment of Alzheimer's disease patients, reducing forgetfulness and other symptoms.

The invention is plant-derived exosomes which is comprise their use in increasing cognitive functions and concentration

In order to fulfil the objectives described above, the invention is exosomes used in the treatment of Alzheimer's disease, cognitive dysfunction, dysmnesia (forgetfulness) and concentration difficulty disorders wherein it comprises contains coffee, ginkgo-biloba and ginseng plants.

The invention is plant-derived exosomes wherein it is in the form of a pharmaceutically usable tablet.

The structural and characteristic features of the invention and all its advantages will be understood more clearly by means of the figures given below and the detailed description written by making references to these figures, so the evaluation should be made taking into account these figures and detailed description.

### DESCRIPTION OF THE FIGURES

FIG. 1: SEM Image of the Exosomes Obtained from Coffee.

FIG. 2: Distribution graph of the Diameters of the Exosomes Obtained from Coffee.

FIG. 3: SEM Image of the Exosomes Obtained from Ginseng.

FIG. 4: Another SEM Image of Exosomes Obtained from Ginseng.

FIG. 5: Distribution Graph of the Diameters of the Exosomes Obtained from Ginseng.

FIG. 6: SEM Image of the Exosomes Obtained from the Ginkgo Biloba.

FIG. 7: Distribution Graph of the Diameters of the Exosomes Obtained from the Ginkgo Biloba.

FIG. 8: A graph showing the effects of Coffee Exosomes applied to HT-22 cells in different concentrations on cell viability.

FIG. 9: A graph showing the effects of Coffee, Ginseng and Ginkgo-Biloba Exosomes applied to HT-22 cells in combination at different concentrations on cell viability.

FIG. 10: A graph showing the effects of different concentrations of Aβ (1-42) on the cell viability of HT-22 cells.

FIG. 11: The is A graph showing the effect of Coffee Exosomes applied in different concentrations to formed HT-22 cells with established Aβ (1-42) toxicity on cell viability.

FIG. 12: A graph showing the effects of ginseng exosomes applied in different concentrations on the cell viability of HT-22 cells.

FIG. 13: A graph showing the effects of Ginseng, Coffee and Ginkgo-Biloba exosomes applied to HT-22 cells in combination at different concentrations on cell viability.

FIG. 14: A graph showing the effect of Ginseng exosomes applied in different concentrations to HT-22 cells with established Aβ (1-42) toxicity on cell viability.

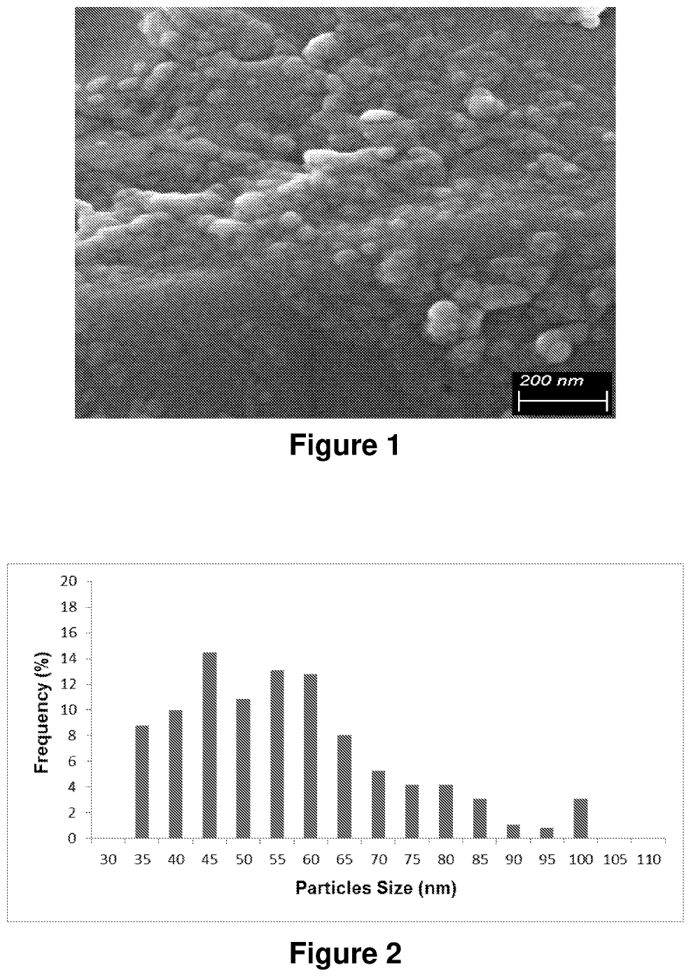

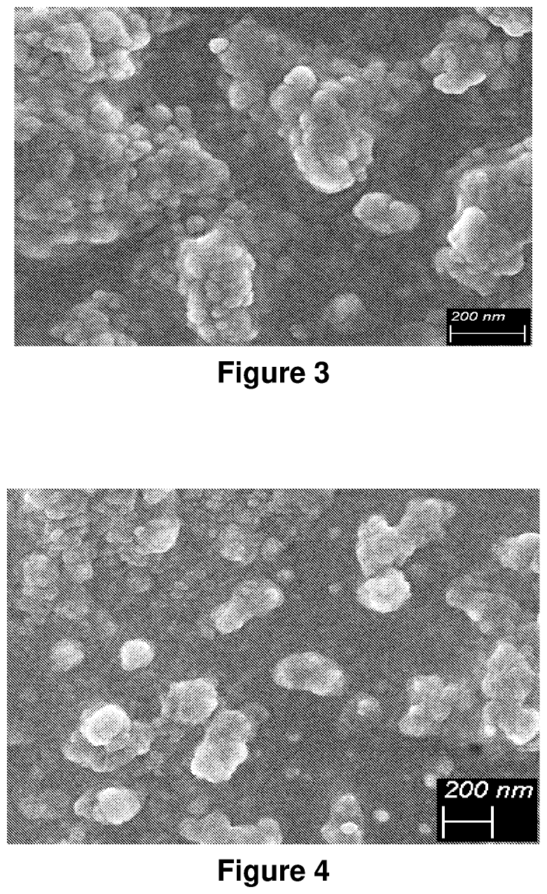

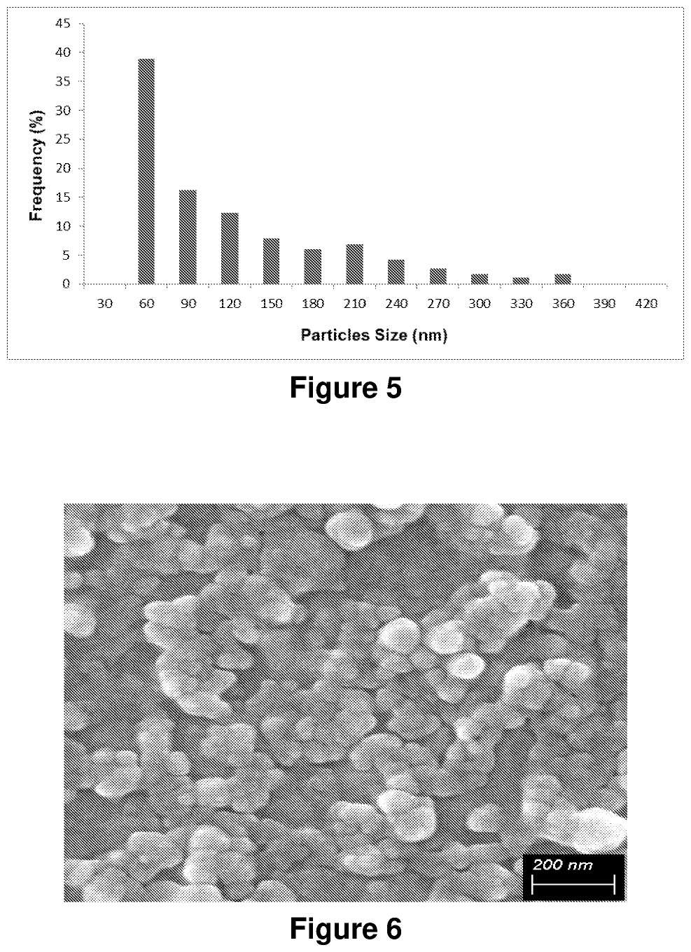

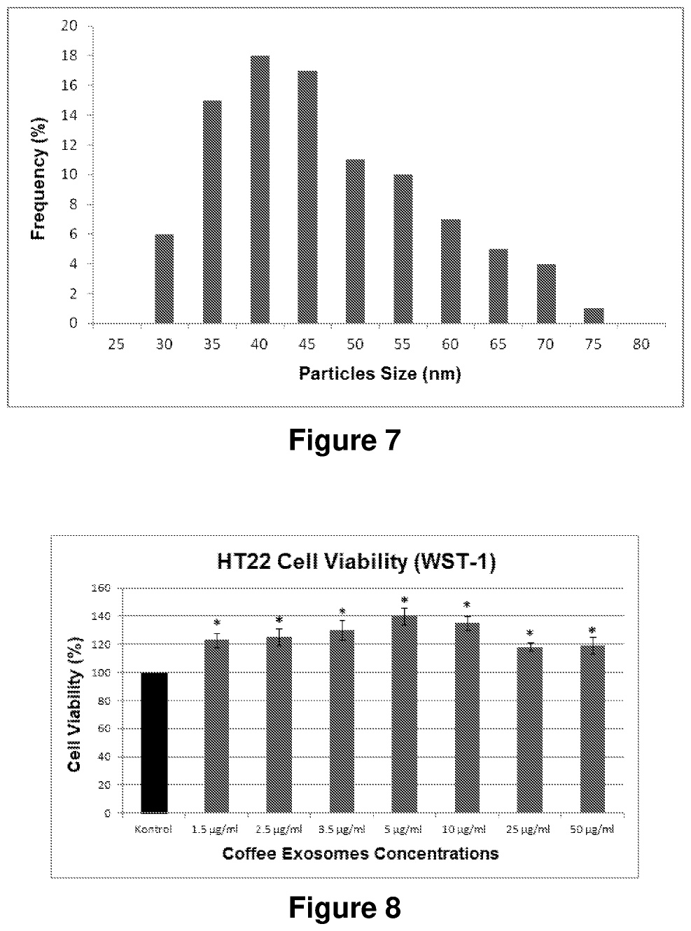

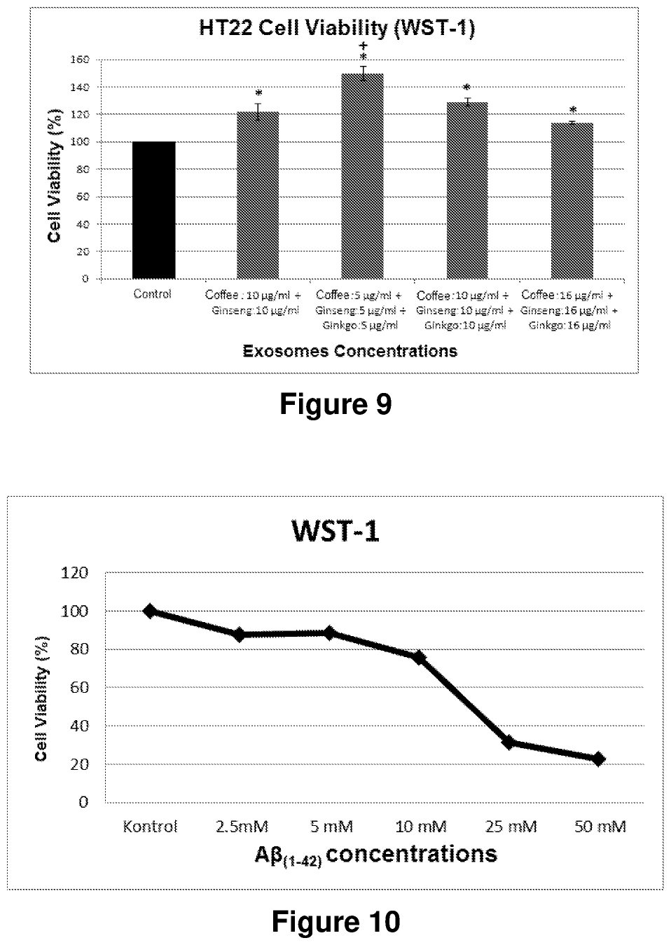

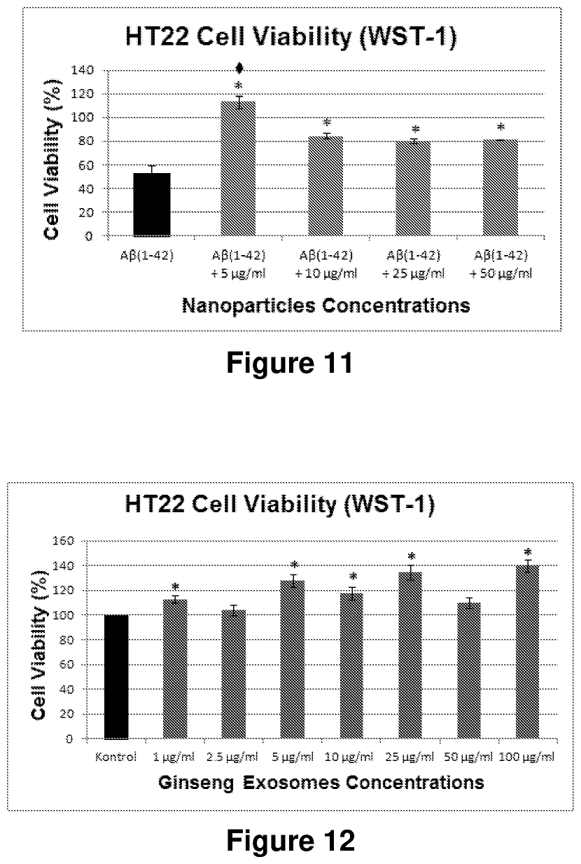

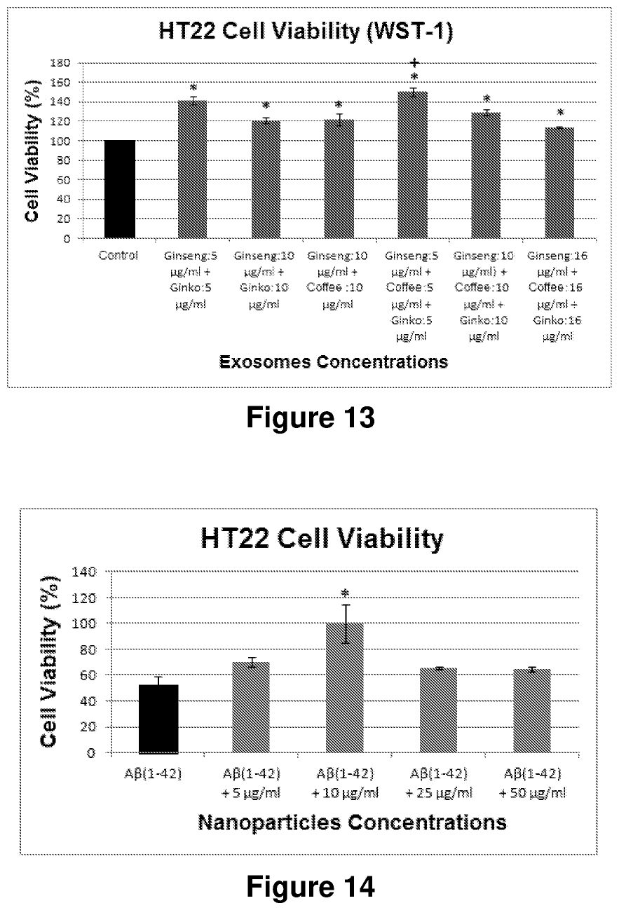

### DETAILED DESCRIPTION OF THE INVENTION

In this detailed explanation, the subject of the invention, plant-derived exosomes obtained from coffee, ginkgo-biloba and ginseng are explained only for a better understanding of the subject and in such a way that they do not create any limiting effects.

The invention relates to the use of coffee, ginkgo-biloba and ginseng plants for obtaining of exosomes.

The invention also relates to the use of exosomes derived from coffee, ginkgo-biloba and ginseng plants in the treatment of Alzheimer's disease, cognitive dysfunction, forgetfulness (dysmnesia) and concentration difficulty disorders.

Exosomes used in the treatment of Alzheimer's disease, cognitive dysfunction, forgetfulness and concentration difficulty disorders comprise coffee, ginkgo-biloba and ginseng plants.

The plant-derived exosomes, which is the subject of the invention, are used especially in the treatment of the disease in Alzheimer's patients, to reduce forgetfulness and other symptoms.

The plant-derived exosomes, which is the subject of the invention, are also used to increase cognitive functions and concentration

According to a preferred embodiment of the invention, the plant-derived exosomes that are the subject of the invention are in the form of a tablet that can be used pharmaceutically.

In the production method of the exosomes used to reduce symptoms and treat the disease in Alzheimer's patients, which is the subject of the invention, differential centrifugation method is used for the isolation of exosomes obtained from coffee, ginkgo-biloba and ginseng.

The production method of the exosomes obtained from coffee, ginkgo-biloba and ginseng, which is the subject of the invention, comprises the following process steps:
i. brewing or mixing of plant extracts and obtaining extract solutions,
ii. centrifugation of solutions in a staged manner at different gravities and durations,
iii. removal of pellets by removing the supernatants obtained after each centrifugation process,
iv. slowly dissolving the resulting pellets obtained after the last centrifuge in 1 ml PBS,
v. transferring the dissolved pellets to the tubes and storing them at about −80° C.

According to a preferred embodiment of the invention, the differential centrifugation stages applied in the process step (ii) of the said production method are as follows, respectively:
a. centrifuging the samples at 200 g for about 10 minutes,
b. centrifuging the resulting supernatants at 2000 g for about 20 minutes,
c. centrifuging the resulting supernatants at 10,000 g for about 30 minutes,
d. centrifuging the resulting supernatants at 150,000 g for about 90 minutes.

The following table-1 shows the properties and effects of the exosomes that are the subject of the invention.

TABLE 1
Properties of Exosomes

| Property | Effect |
|---|---|
| Small size | Ability to penetrate further through the blood |
| | brain barrier |
| Nano-sized | Easy access to the areas of the brain responsible |
| transportation | for memory |
| Biocompatibility | The use of natural products with very low toxicity |
| | and side effects instead of synthetic molecules in |
| | the treatment of Alzheimer's |
| Treatment of | Therapeutic effects |
| Alzheimer's disease | |

The experimental studies related to the invention are described in detail below:

### Isolation of Exosomes by Differential Centrifugation Method:

The samples were centrifuged at 200 g for 10 minutes, the pellets were discarded by taking the supernatants obtained after centrifugation. The resulting supernatants were centrifuged for 20 minutes in 2000 g, the pellets were discarded by taking the supernatants obtained after centrifugation. The resulting supernatants were centrifuged at 10,000 g for 30 minutes, and the pellets were discarded by taking the supernatants obtained after centrifugation. The resulting supernatants were centrifuged at 150,000 g for 90 minutes, the supernatants obtained after centrifugation were discarded and the obtained pellets were slowly dissolved in 1 ml PBS and transferred to Eppendorf tubes. The samples were stored at −80° C. to be kept until the work day.

### SEM (Scanning Electron Microscope) Analysis and Determination of the Diameter Distributions of Exosomes:

The diameter distribution of the exosomes obtained from coffee, ginkgo-biloba and ginseng was analyzed by SEM. SEM analysis is one of the best methods for obtaining quantitative and qualitative information about porous structures. It is widely used in determining the average pore size and distribution. First of all, the exosomes were washed with PBS solution and fixed with 2.5% glutaraldehyde solution. Then the samples were washed with PBS and glutaraldehyde was removed. Samples were dehydrated with increasing ethanol concentrations from 30% to 100%. Then, the samples were dried at room temperature and photographed with high resolution in different positions with appropriate magnification. The distribution of the diameters of the exosomes in the obtained images was measured with the help of the Image J program (randomly selected in the SEM grid).

### Bradford Protein Quantity Determination Method:

The standards were prepared in Eppendorf tubes according to the Bradford Protein Assay Kit procedure. From the prepared standards, 5 μL was added to each well from exosome samples obtained from coffee, Ginkgo biloba and ginseng. Then, 200 ML of Bradford Reagent Solution was added to each well and pipetting was performed. The standard and samples were incubated at room temperature for 15 min. The absorption values of the standard and samples were measured at a wavelength of 570 nm using a Microplate Reader. A standard curve was drawn according to the absorption values obtained from the standards and the amount of protein was calculated for each sample.

### HT-22 Cell Culture and WST-1 Cell Viability Test:

The cells were allowed to multiply at 37° C. in an environment with 95% humidity and 5% CO₂ at the feed yard where 10% fetal bovine serum (Invitrogen), 2 mm glutamine, 100 units/ml penicillin and 100 mg/ml streptomycin were used together with DMEM (Dulbecco's Modified Eagle's Medium) (pH: 7.2-7.4). Cell viability and number were determined using a hemocytometer device with 0.4% trypan blue dye. Non-living cells absorb the trypan blue dye and appear on a light microscope with their nuclei stained blue. Thus, non-living cells could be distinguished from living cells and counted. In all experiments, the live cell ratio was determined as (Live cell ratio (%)=Number of cells that have not received dye/total number of cells×100) before starting the experiments, and experiments were started at values where the cell viability was 95%. HT-22 cells were incubated with exosomes obtained from coffee, ginkgo-biloba and ginseng at concentrations of 1-50 μg/ml for 24 hours. After 24 hours of incubation, the cell viability of HT-22 cells was analyzed with the WST-1 test. Before the WST-1 viability test was performed, WST-1 stock solution was prepared and 100 μl solution was applied to each well from the stock solution. The cells were then incubated for 2 hours. After 2 hours of incubation, the absorbance values were measured with a Microplate Reader at a wavelength of 450 nm.

Setting Up an In-Vitro Alzheimer's Model with Aβ (1-42) Toxicity:

The lyophilized Aβ (1-42) peptides were dissolved in distilled water and DMSO and a 1 MM stock solution was prepared. The prepared stock solution was diluted with series dilutions by preparing Aβ (1-42) solutions in different contractions (2.5 μM, 5 μM, 10 μM, 25 μM and 50 μM). HT-22 cells were planted in plates of 96 with 10⁴ cells/ml in each well. The cells were incubated with Aβ (1-42) peptides of different concentrations (5 μM, 5 μM, 10 μM, 25 μM and 50 μM) for 24 hours. After incubation, the WST-1 cell viability test was performed to determine the cell viability of the cells and the effective dose value of Aβ (1-42) was determined. In the in-vitro Alzheimer's model we created with Aβ (1-42) toxicity at the effective dose value in HT-22 cells, the cells were incubated with exosomes derived from ginseng and coffee at concentrations of 5-50 μg/ml for 24 hours, and the cell viability of the cells was determined after 24-hour incubation.

The results of the experimental studies are described below:

It has been observed that coffee exosomes at different concentrations (1-50 μg/ml) applied alone or in combination with ginkgo-biloba and ginseng exosomes significantly increased the cell viability of hippocampal neuron cells statistically compared to the cell viability of control group cells without exosome application (p<0.05). Similarly, it has been observed that ginseng exosomes at different concentrations (1-50 μg/ml) applied alone or in combination with coffee and ginkgo-biloba exosomes significantly increased the cell viability of hippocampal neuron cells compared to the cell viability of control group cells without exosome application (p<0.05).

In the second stage of the study, an in-vitro Alzheimer's model was created with Aβ (1-42 toxicity in HT-22 cells. Exosomes at concentrations of 1-50 μg/ml obtained from coffee and ginseng were applied to cells with induced Aβ (1-42) toxicity and the changes in the viability of their cells were analyzed. Exosomes obtained from coffee at concentrations of 1-50 μg/ml significantly increased the cell viability of the cells with Aβ (1-42) toxicity. (p<0.05). Exosomes obtained from ginseng at a concentration of 10 μg/ml significantly increased the cell viability of cells with Aβ (1-42) toxicity (p<0.05).

In summary, exosomes obtained from coffee, ginkgo-biloba and ginseng significantly increased cell proliferation both in healthy hippocampal neuron cells and in neuron cells in the hippocampal region of the brain related to memory, where Aβ (1-42) toxicity has been established.

These results show that exosomes obtained from coffee, gingko-biloba and ginseng can be used to treat the following;
In Alzheimer's disease, the treatment of the disease and the reduction of symptoms, and
Disorders of cognitive dysfunction, forgetfulness (/dysmnesia) and concentration difficulty.

Exosomes obtained from coffee, gingko-biloba and ginseng have much higher ability to penetrate the blood-brain barrier due to their small size. By means of these properties, exosomes derived from coffee, gingko-biloba and ginseng have the potential to show much higher biological activity than large-sized structures such as extracts. Exosomes derived from coffee, gingko-biloba and ginseng, which is the subject of the invention, will be much more effective in the treatment of Alzheimer's disease and in the treatment of cognitive dysfunction, forgetfulness and concentration difficulties than existing coffee, gingko-biloba and ginseng extracts.

1. Obtaining of exosomes from coffee, and/or ginkgo-biloba and and/or ginseng plants.

2. Use of exosomes obtained from coffee, and/or ginkgo-biloba and/or ginseng plants in the treatment of Alzheimer's disease, cognitive dysfunction, dysmnesia and poor concentration disorders.

3. The use according to claim 2, wherein the plant-derived exosomes are used in the treatment of Alzheimer's disease and the reduction of forgetfulness and other symptoms

4. The use according to claim 2, wherein the plant-derived exosomes are used in improving cognitive functions and concentration.

5. Exosomes used in the treatment of Alzheimer's disease, cognitive dysfunction, forgetfulness and poor concentration disorders, wherein the exosomes are obtained from coffee, and/or ginkgo-biloba and/or ginseng plants.

6. The use according to claim 2, wherein the plant-derived exosomes are in the form of a pharmaceutically usable tablet.
